(19) United States
(12) Patent Application Publication
(10) Pub. No.: US 2026/0108287 A1
(43) Pub. Date: Apr. 23, 2026

(54) STRIKING APPARATUS FOR A MEDICAL EXTRACTING TOOL

(71) Applicant: Shukla Medical, St. Petersburg, FL (US)

(72) Inventors: Mitchell Ryan Kiska, Seminole, FL (US); Mohamad El Akhal, Clearwater, FL (US)

(73) Assignee: Shukla Medical, St. Petersburg, FL (US)

(21) Appl. No.: 18/923,955

(22) Filed: Oct. 23, 2024

(51) Int. Cl. A61B 17/92; A61B 17/00; A61B 17/88

(52) U.S. Cl. CPC A61B 17/92; A61B 17/8886; A61B 2017/00473

(57) ABSTRACT

A striking apparatus and method is described for enhancing the surgical removal of medical devices, such as surgical screws and the like. The striking apparatus may include an elongated body that may include an annular distal end, a neck extending from the elongated body; and a fastener extending from the neck. The elongated body may include a first tapering portion extending from the annular distal end and a second tapering portion extending from the first tapering portion. The first tapering portion may be an annular first tapering portion. The striking apparatus may detachably couple with a medical extractor, thus providing additional force to the extracting mechanism provided by the extracting tool.

### FIELD OF THE INVENTION

The present invention relates to devices and methods useful for aiding in the extraction of an implanted medical device from a patient.

### BACKGROUND OF THE DISCLOSURE

Medical devices, such as medical screws and the like may be implanted within a patient. At some point the medical devices may need to be removed from the patient via a medical extraction tool. The efficient removal of the medical devices can reduce trauma to a patient's tissues and the time required for removal, which in turn can reduce time under anesthesia. In some instances, there is a need for a tool to assist a medical extraction tool in the removal of the medical devices. Such tools may include a striking apparatus that can be coupled to and removed from an extraction tool.

### SUMMARY OF THE DISCLOSURE

A striking apparatus is described for enhancing a medical extracting tool in the removal of medical devices, such as surgical screws and the like. As described further herein, the striking apparatus can detachably couple with a medical extractor, thus providing additional force to the extracting mechanism(s) provided by the extracting tool.

In an aspect, the present disclosure provides a striking apparatus for a medical extraction tool. The striking apparatus may include an elongated body. The elongated body may have an annular distal end, a neck extending from the elongated body; and a fastener extending from the neck.

The elongated body may include a first tapering portion extending from the annular distal end and a second tapering portion extending from the first tapering portion. The first tapering portion may be an annular first tapering portion. The first tapering portion may be frustum shaped. The first tapering portion may be positioned about a midpoint of an overall length of the striking apparatus. The second tapering portion may have an overall diameter less than the first tapering portion. An outer surface profile of the first tapering portion and the second tapering portion may include a point of inflection.

In an aspect, the elongated body may have an overall diameter of about 30 mm to about 33 mm. The annular distal end may have a wall thickness of about 9 to about 11 mm. The wall may be configured with such thickness to permit the striking tool to withstand the force acted upon it. The elongated body may have an overall longitudinal length of about 95 mm to about 100 mm.

In an aspect, the neck may include a flat side surface. The neck may be square-shaped, hex-shaped, or circular-shaped. The neck may be configured to receive a tool to manipulate the striking apparatus about a longitudinal axis of the elongated body of the striking apparatus. For example, the neck may be configured to receive a tool (e.g., a wrench) that may turn the striking tool about a longitudinal axis of the elongated body thereby resulting in the striking apparatus further engaging the extraction tool or further disengaging the extraction tool. The neck may include a plurality of ridges, knurlings, and/or protrusions.

In an aspect, the fastener may include threads, a quick connect, a taper-lock, a taper, a cylinder, and/or a cam lock. The fastener comprises a proximally facing flat end having a female socket. The female socket is a linear socket, a hex socket, a Philips socket, or a Torx socket.

In an aspect, the elongated body, neck and fastener may form a unitary part. The striking apparatus may be formed from a subtractive manufacturing process (e.g., machining), a cast manufacturing process, and/or an additive manufacturing process (e.g., 3D printing). The striking apparatus may comprise stainless steel. The striking apparatus may have a maximum weight of about 1.75 kilograms. In an example, the striking apparatus may have a longitudinal midplane and a weight of the striking apparatus is substantially balanced about the midplane. In an aspect, the annular distal end of the elongated body may include an aperture. The elongated body may be substantially hollow.

In an aspect, a medical extraction tool may include an extractor body having an attachment mechanism and a striking apparatus. The striking apparatus may include an elongated body having an annular distal end; a neck extending from the elongated body; and a fastener extending from the neck. The fastener may extend from the neck of the striking apparatus and be configured to detachably couple with the attachment mechanism. The medical extraction tool may include a first arm extending from the extractor body and having a first jaw; and a second arm pivotally connected to the first arm and having a second jaw.

In an aspect, the elongated body of the striking apparatus may include a first tapering portion extending from the annular distal end and a second tapering portion extending from the first tapering portion. The first tapering portion may be an annular first tapering portion. The attachment mechanism about the tool body may be an orifice.

In an aspect, the fastener of the striking apparatus may include threads, a quick connect, a taper-lock, a taper, a cylinder, and/or a cam lock. The fastener may be configured to fastenly couple to the extraction tool upon the neck of the striking apparatus being turned about a longitudinal axis of the elongated body. The fastener may include a proximally facing flat end having a female linear socket, hex socket, Philips socket, and/or Torx socket.

### BRIEF DESCRIPTION OF THE FIGURES

The foregoing summary, as well as the following detailed description of the exemplary embodiments of the subject disclosure, will be better understood when read in conjunction with the appended drawings. For the purpose of illustrating the present disclosure, exemplary embodiments are shown in the drawings. It should be understood, however, that the subject application is not limited to the precise arrangements and instrumentalities shown.

FIG. 1 is a perspective view of an example striking apparatus, as described herein;

FIG. 2 is a side view of the striking apparatus of FIG. 1;

FIG. 3 is a front view of the striking apparatus of FIG. 1;

FIG. 4 is a back view of the striking apparatus of FIG. 1;

FIG. 5 of a perspective cross-sectional view of the striking apparatus of FIG. 1;

FIG. 6 is a longitudinal cross-sectional view of the striking apparatus of FIG. 1;

FIG. 7 is a perspective view of the striking apparatus of FIG. 1 coupled with an example medical extraction tool;

FIG. 8 is an exploded perspective view of the striking apparatus and the example medical extraction tool of FIG. 7;

FIG. 9 is another exploded perspective view of the striking apparatus and the example medical extraction tool of FIG. 7; and

FIG. 10 is a top plan view of the striking apparatus coupled with the example medical extraction tool of FIG. 7.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one,” and “one or more,” as used in the specification, means at least one but may include any number above one.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

Medical extracting tools may be used during medical procedures, such as revision procedures intended to replace or compensate for a failed, expired, or no longer needed medical implant. In examples the medical extracting tool may be used to remove one or more types of devices from a patient, such as IM nail implants, knee implants, hip implants, pins, k-wires, nuts, spine rods, staples, screws, and the like. Medical extracting tools may be made in various configurations and may include one or more types of attachments. For example, medical extraction tools may include one or more configurations having one or more jaws, hubs, and/or striking devices.

Striking tools (e.g., striking apparatuses) may be used to provide a force to the medical extraction tool. The force provided to the extraction tool by the striking apparatus may be provided (e.g., originated) by a tool, such as a hammer, mallet, and the like, exerting the force to the striking apparatus which then transfers the force to the medical extraction tool. In other examples a hand may provide force to the striking apparatus, and the striking apparatus may transfer such force to the medical extraction tool.

In some examples the striking apparatus may receive a force that is transverse to the body of the striking apparatus, although in other examples the force may be longitudinal to the body of the striking apparatus. The striking apparatus may be coupled to the medical extraction tool such that the force received by the striking apparatus transferred from the striking apparatus to the medical extraction tool and continues along the same axis in which the force was received by the striking apparatus. For example, the striking apparatus may be coupled to the medical extraction tool such that the force received by the striking apparatus is longitudinal to the medical extraction tool, perpendicular to the medical extraction tool, etc., and the force transferred from the striking apparatus to the medical extraction tool continues along the same axis. In other examples the force may be provided to the striking apparatus in one axis, and the force from the striking apparatus may be transferred to the medical extraction tool in another axis.

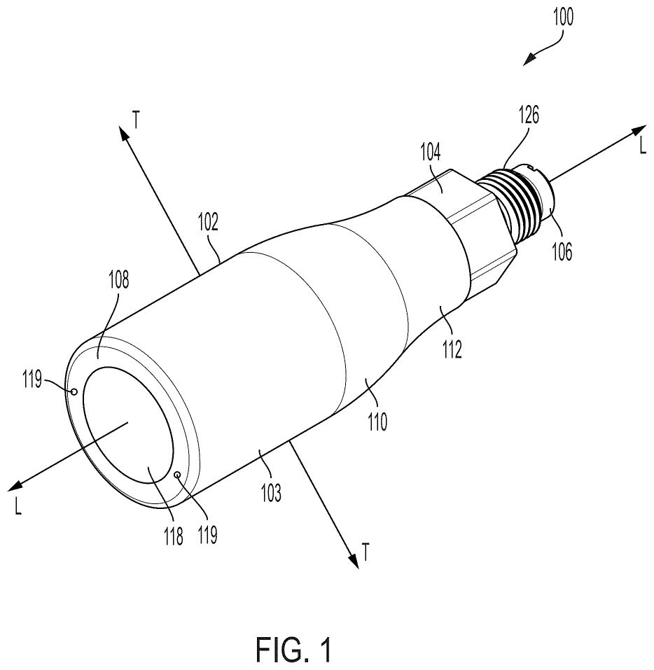

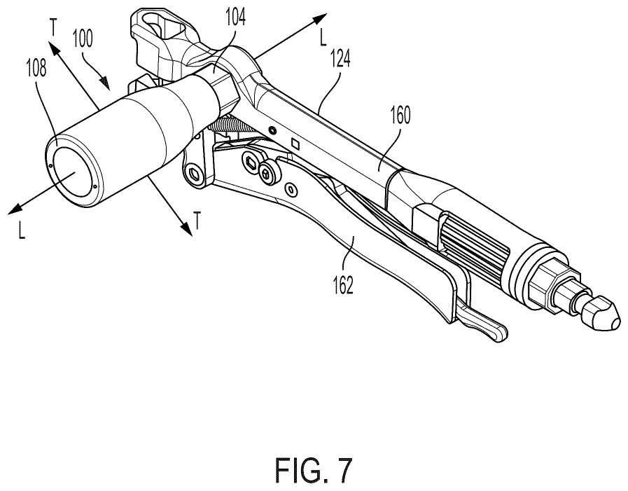

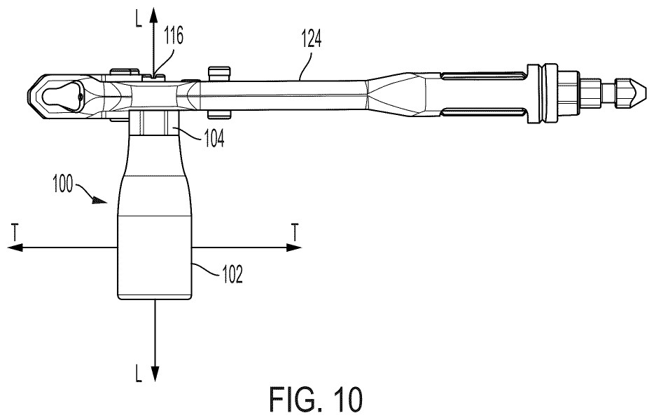

Referring now to the drawings, FIG. 1 shows an example striking apparatus 100. In examples the striking apparatus 100 may be detachably coupled to a medical extraction tool (such as extraction tool 124 (FIGS. 7 and 10)). In other examples the striking apparatus 100 may be fixedly attached to an extraction tool. As described herein, the striking apparatus 100 may be used to provide a force to the medical extraction tool in one of a variety of axes to the medical extraction tool. Such force may be used to assist in the removal of a medical implant or other hardware to be removed from a patient.

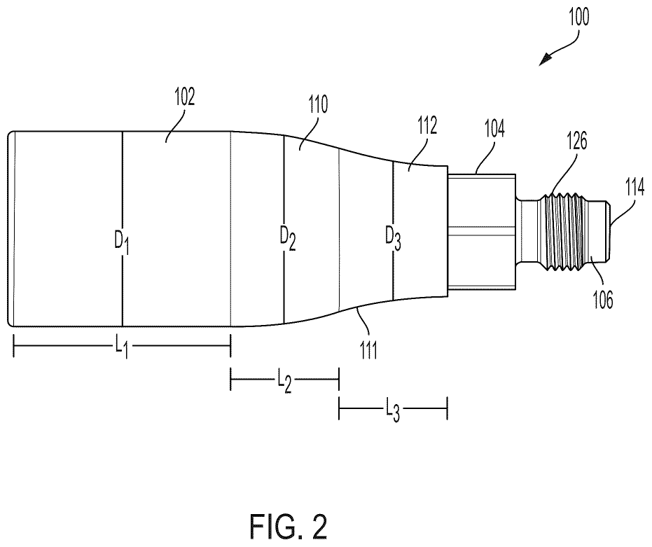

As shown in FIGS. 1 and 2, striking apparatus 100 may include one or more portions. For example, striking apparatus 100 may include an elongated body 102, neck 104, and fastener 106. One or more of the elongated body 102, neck 104, and fastener 106 may form a single unitary part e.g., be of unitary construct or of unitary construction. In other examples, one or more of the elongated body 102, neck 104, and fastener 106 may be separately joined to one another. For example, in examples elongated body 102 may be a piece separate from the neck 104 and the fastener 106 and joined with the neck 104 and the fastener 106. In other examples the elongated body 102 may be unitary piece formed with the neck 104, and the fastener 106 may be a separate piece joined with the neck 104 and the body 102.

One or more (e.g., all) portions of striking apparatus 100 may be formed of a metal, such as steel or stainless steel (e.g., heat treated 17-4 stainless steel). Stainless steel may be used to enhance impact resistance to striking apparatus 100. For example, stainless steel may be used to enhance resistance to impact upon striking apparatus 100 via a striking tool, such as a mallet, hammer, and the like. In examples one or more portions of striking apparatus may be formed of other material, such as plastic, metals other than stainless steel (e.g., iron, brass, titanium, copper, steel, aluminum), and the like. For example, body 102 may be formed of stainless steel and one or more of first tapering portion 110 and second tapering portion 112 may be formed of materials other than stainless steel (e.g., plastic, brass, titanium, copper, steel, aluminum).

Body 102 may include one or more portions, such as an open or annular distal end 108, an annular portion 103, a first tapering portion 110, a second tapering portion 112, and a proximal end 120. Body 102 is an elongated body that includes a first tapering portion 110 extending from the annular distal end 103 and a second tapering portion 112 extending from the first tapering portion 110. First tapering portion 110 may be positioned about a midpoint of an overall length of the striking apparatus. A profile (e.g., an outer surface profile) of the first tapering portion 110 and the second tapering portion 112 includes a point of inflection 111. First tapering portion 110 may be an annular first tapering portion. One or more of the first and second portions may be frustrum shaped. For example, first and second tapering portions 110, 112 are frustum shaped.

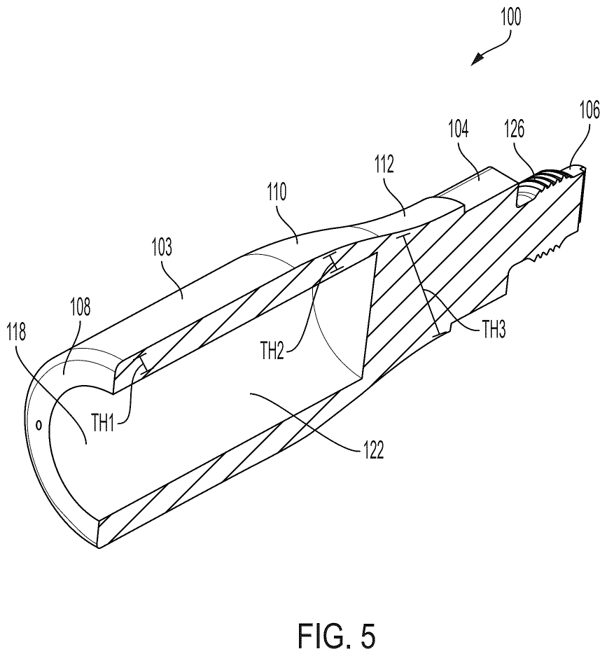

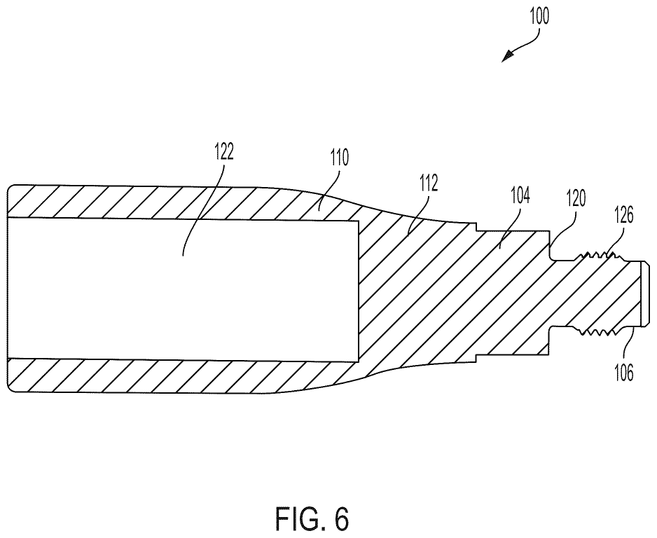

As best shown in FIGS. 5 and 6, the body 102 has a hollow interior 122, although in other examples the body 102 may be solid (e.g., completely solid), or porous, or of another structural configuration, such as honeycomb, enclosed, layered, etc. Body 102 may include a hollow interior 122 to reduce costs of material necessary in manufacturing the striking apparatus 100, reduce weight of the striking apparatus, etc. The hollow interior 122 may be of various depths. For example, the hollow interior may extend from the annular distal end 108 to the first tapering portion 110, from the annular distal end 108 to the second tapering portion 112, from the annular distal end 108 to the annular proximal end 108, from the first tapering portion 110 to the second tapering portion 112, or any length in between.

Alternatively expressed, the body 102 is a hollow interior that is counterbored to form a counterbore recess. The counterbore recess may be a flat edged (e.g., square shaped, hexagonal shaped, etc.) counterbore recess, or any other shape suitable for its intended purpose. As shown on FIG. 5, hollow interior 122 may extend to about ⅔ of the overall longitudinal length of the body 102, although in examples hollow interior 122 may be ⅓ of the overall longitudinal length of body 102, half of the body, or hollow interior 122 may extend to any length of the body suitable for its intended purpose. In examples hollow interior 122 may extend about 50-70% of a longitudinal length from annular distal end 108.

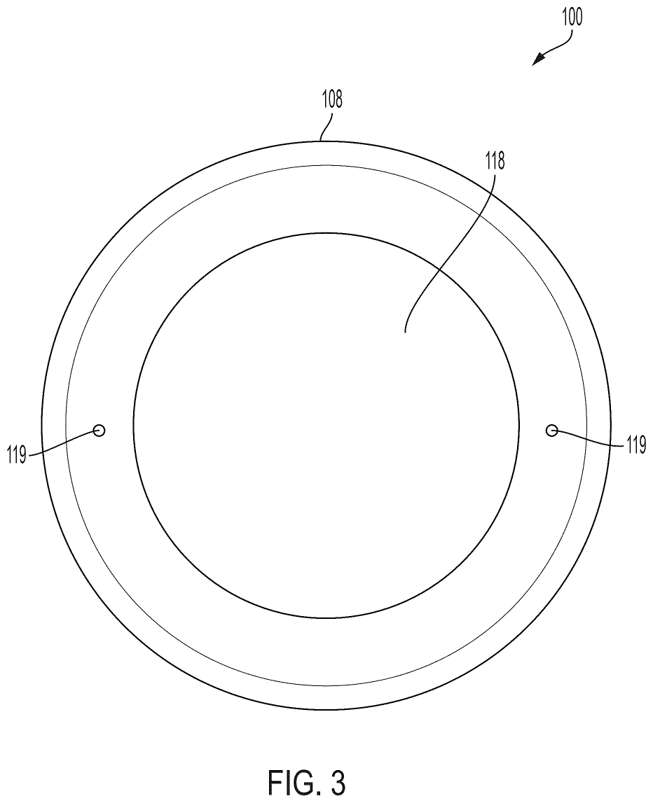

One or more apertures may provide access to hollow interior 122. For example, as shown in FIGS. 1 and 3, annular distal end 108 may include an aperture 118. In examples in which body 102 includes a hollow interior (e.g., hollow interior 122), aperture 118 provides access to the hollow interior. Aperture 118 may be of varying sizes e.g., of varying diameters. For example, the aperture 118 may have a diameter that is less than half the diameter of annular distal end 108, aperture 118 may have a diameter that is half the diameter of annular distal end 108, or aperture 118 may have a diameter that is more than half the diameter of annular distal end 108. For example, aperture 118 may have a diameter that is 40%, 50%, 60%, 70%, 80%, or 90% of the total diameter of annular distal end 108. The annular distal end 108 may have a wall thickness of about 8 to about 11 mm. Striking apparatus 100 is configured to have a weight or mass of about 0.5 kilograms to 1.75 kilograms. In examples, striking apparatus 100 is configured to have a minimum weight of about 0.5 kilograms and a maximum weight of about 1.75 kilograms.

Annular portion 103, first tapering portion 110, and second tapering portion 112 may include diameters (e.g., overall diameters or outside diameters) that may be the same as one another or different from one another. As shown in FIG. 2, annular portion 103 may have a diameter D1, first tapering portion 110 may have a diameter D2, and second tapering portion 112 may have a diameter D3. As shown on FIG. 2 for D1, when the portion is annularly shaped (and not tapered) the diameter D1 remains consistent throughout the length of the portion. Conversely, as shown in FIG. 2 for D2 and D3, when a portion is tapered, the diameter may increase or decrease throughout the length of the portion.

First tapering portion 110 may have a diameter (e.g., overall diameter) that is smaller than annular portion 102. Second tapering portion 112 may have a diameter (e.g., overall diameter) that is less than first tapering portion 110. Although FIG. 2 shows the diameters going from larger at the distal end to smaller at the proximal end, such examples are for illustration purposes only. In other examples diameters may go from smaller at the distal end to larger at the proximal end.

As shown in FIG. 2, annular portion 103 may have a larger diameter D1 than first tapering portion 110 and second tapering portion 112. In examples the portions can be configured with varying diameters. For example, first tapering portion 110 may have a diameter that is greater than the diameter of annular portion 103. First tapering portion 110 may have a diameter that changes throughout the first tapering portion 110. In such example, a diameter of a portion of first tapering portion 110 may be greater than the diameter of annular portion 103 and another portion of first tapering portion 110 may be smaller and/or the same as the diameter of annular portion 103. Body 102 may have an overall diameter of about 30 mm to about 33 mm.

Each of annular portion 103, first tapering portion 110, and second tapering portion 112 may have differing lengths. In some examples annular portion 103, first tapering portion 110, and second tapering portion 112 may have the same lengths, whereas in other examples one or more of annular portion 103, first tapering portion 110, and second tapering portion 112 may have differing lengths. For example, as shown in FIG. 2 annular portion 103 may have a length L1 that is longer than a length L2 of first tapering portion 110 and a length L3 of second tapering portion 112, whereas in other examples annular portion 103 and first tapering portion 110 may have lengths that are shorter than second tapering portion 112. Body 102 may have an overall longitudinal length of about 95 mm to about 100 mm.

The length of body 102 (overall longitudinal length) may be greater than one or more diameters of annular portion 103, first tapering portion 110, and second tapering portion 112. For example, the length of body 102 (i.e., L1+L2+L3) may be greater than the largest diameter of annular portion 103, first tapering portion 110, or second tapering portion 112. Using the example shown in FIG. 2, the length of body 102 (i.e., L1+L2+L3) may be larger than the diameter of annular portion 102 (i.e., D1), the length of body 102 (i.e., L1+L2+L3) may be larger than the diameter of first tapering portion 110 (i.e., D2), and the length of body 102 (i.e., L1+L2+L3) may be larger than the diameter of second tapering portion (i.e., D3).

Aperture 118 may include attachment mechanism 119 that can attach a device to striking apparatus 100 via such attachment mechanism. As an example, aperture 118 may include one or more inserts, inside threads, clips, or the like as an attachment mechanism 119 in which another device can use to attach to the striking apparatus 100. As an example, an extension can be attached to the striking apparatus 100 via attachment mechanism, which may lengthen the body 102 of the striking apparatus 100. In other examples, other tools, such as gripping, grabbing, vibrating, and the like, may be attached to striking apparatus 100 via attachment mechanism found within aperture 118.

One or more of annular portion 103, first tapering portion 110, and second tapering portion 112 may be shaped having straight edges (e.g., may be square shaped, hexagon shaped, etc.). In other examples, annular portion 103 may be tapered. In further examples the first tapering portion 110 may be annularly shaped. In still other examples, the annular portion 103, the first tapering portion 110, and the second tapering portion 112 may all be annularly shaped or may all be tapered. Although FIG. 1 shows three portions (e.g., annular portion 103, first tapering portion 110, and second tapering portion 112), in alternative exemplary embodiments the striking apparatus may include less than three portions, more than three portions, or exactly three portions, in which one or more of the portions may be one or annularly shaped, flat-edged shaped, tapered, and the like.

Striking apparatus 100 may include one or more portions extending from body 102. As shown in FIGS. 1 and 2, neck 102 may extend from body 102 and fastener 106 may extend from neck 104. In other examples neck 104 and fastener 106 may separately extend from body 102. In still other examples neck 104 and fastener 106 may be the same portion (e.g., neck 104 may include neck 104 and fastener 106).

As shown in FIGS. 1 and 7, neck 104 may be used to turn the striking apparatus 100 along a longitudinal axis L of striking apparatus 100 (e.g., along longitudinal axis L of body 102). For example, a gripping means (e.g., wrench, fingers) may grab hold of neck 104 and turn striking apparatus 100 around longitudinal axis L via neck 104.

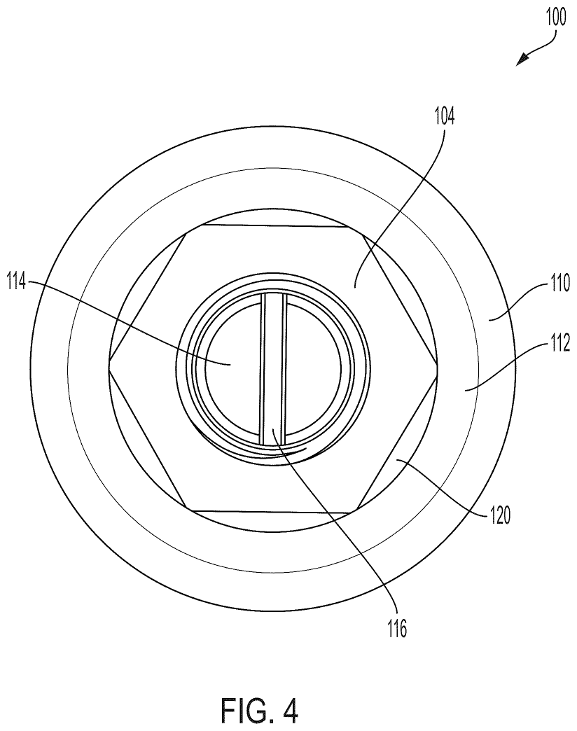

Neck 104 may be formed in many shapes. In some examples neck 104 may include one or more flat surfaces, whereas in other examples neck 104 may include one or more curved surfaces. In examples neck 104 may include a combination of flat surfaces and curved surfaces. Neck 104 may include one or more protuberances. An attachment mechanism may attach to the protuberance to move the striking apparatus 100 about the longitudinal axis L. Neck 104 may include one or more orifices or inserts in which an attachment mechanism may attach to for moving the striking apparatus 100 about the longitudinal axis L. In examples, neck 104 may be square-shaped, hex-shaped (FIG. 4), circular-shaped, or the like. Neck 104 may include one or more structures, e.g., ridges, knurlings, or protrusions to aid in the gripping of neck 104.

Striking apparatus 100 may include a fastener, such as fastener 106 shown on FIGS. 1 and 2. Fastener 106 may extend from neck 104, although in examples fastener 106 may be formed from elongated body 102 or fastener 106 may be formed from neck 104. In examples fastener 106 may extend from other portions, such as from elongated body 102 or another portion. Fastener 106 may be used to couple (e.g., detachably couple) striking apparatus 100 with one or more other devices. For example, fastener 106 may be used to couple striking apparatus 100 with a medical extractor tool (such as extractor tool 124 shown on FIG. 7). In other examples fastener 106 may be fixedly couple (i.e., permanently couple) striking apparatus 100 to extractor tool 124. For example, striking apparatus 100 may be formed as a unitary piece with extractor tool 124. Extractor tool 124 may have one or more arms. For example, extractor tool 124 may have a first arm 160 extending from the extractor body and including a first extraction means (e.g., a first jaw) and a second arm 162 pivotally connected to the first arm and including a second extracting means (e.g., second jaw).

Striking apparatus 100 may be used to provide a force to the medical extraction tool 124. The force provided to the extraction tool 124 by the striking apparatus 100 may be provided by a tool, such as a hammer, mallet, and the like, exerting the force upon the striking apparatus 100 which then transfers the force to the medical extraction tool 124. In other examples a hand may provide force to the striking apparatus 100, and the striking apparatus 100 may transfer such force to the medical extraction tool 124. For example, as shown on FIG. 1 and FIG. 7, a force may be applied to the striking apparatus 100 along one or more axes. For example, a force may be applied to the striking apparatus 100 along one or more axes transverse to striking apparatus 100 (e.g., one or more transverse axes of body 102). In examples in which body 102 is annularly shaped, the force may be applied to striking apparatus 100 along any of the transverse axes along the annularly shaped body 102. In examples in which body 102 is flat sided (e.g., square shaped, hexagon shaped, etc.), the force may be applied to striking apparatus 100 along any of the transverse axes along the flat sided body 102.

Body 102 (e.g., one or more portions of body 102, such as one or more of annular portion 103, first tapering portion 110, and/or second tapering portion 112) may have a wall of a thickness configured to absorb the force exerted along the transverse axis T (see FIGS. 1 and 7) of the body 102. As shown in FIG. 5, the annular portion 103 may have a wall thickness TH1 that may be about 9 mm to about 11 mm, the first tapering portion 110 may have a wall thickness TH2 that may be about 4 mm to about 6 mm, and/or the second tapering portion 112 may have a wall thickness TH3 that may be about 20 to about 25 mm. The force applied along the transverse axis T may be provided to any number of portions of body 102. For example, a force may be provided along the transverse axis T to a side portion of body 102, a bottom portion of body 102, a top portion of body 102, or any surface of body 102 in between the side, top, and bottom of body 102. The force provided along the transverse axis T may be provided to annular portion 103 of striking apparatus 100, first tapering portion 110, and/or second tapering portion 112. In other words, the force applied to the body may be applied at any angle or at angles covering 360 degrees relative to its longitudinal axis.

In addition to, or in the alternative of, applying a force along the transverse axis T a force may be applied to striking apparatus 100 along the longitudinal axis L of striking apparatus 100 (see FIGS. 1 and 7). The force may be applied to the striking apparatus 100 via a hammer, mallet, hand, and the like. The force exerted upon the striking apparatus 100 may be transferred to the extraction tool 124 along the transverse axis T of striking apparatus 100, the longitudinal axis L of striking apparatus 100, or any axis of striking apparatus in which there is surface area in which the force can be applied. In other words, the striking apparatus allows a user to strike the body at an unlimited number of angles, or unconstrained angles or directions.

As described herein, in some examples the striking apparatus may be coupled to the extraction tool 124 such that the force received by the striking apparatus is along the transverse axis T and/or the longitudinal axis L and the force transferred from the striking apparatus 100 to the extraction tool 124 may continue in the respective transverse axis T and/or longitudinal axis L. In other examples the striking apparatus 100 may be coupled to the extraction tool 124 such that the force received by the striking apparatus is along an axis that is other than along the transverse axis T or the longitudinal axis L. For example, the striking apparatus 100 may be coupled to the extraction tool 124 such that the force received by the striking apparatus 100 is along an axis other than the transverse axis T or the longitudinal axis L, and the force transferred from the striking apparatus 100 to the extraction tool 124 continues along the axis that is other than the transverse axis T or the longitudinal axis L. In other examples the force may be provided to the striking apparatus in one axis, and the force from the striking apparatus may be transferred to the extraction tool 124 in another axis.

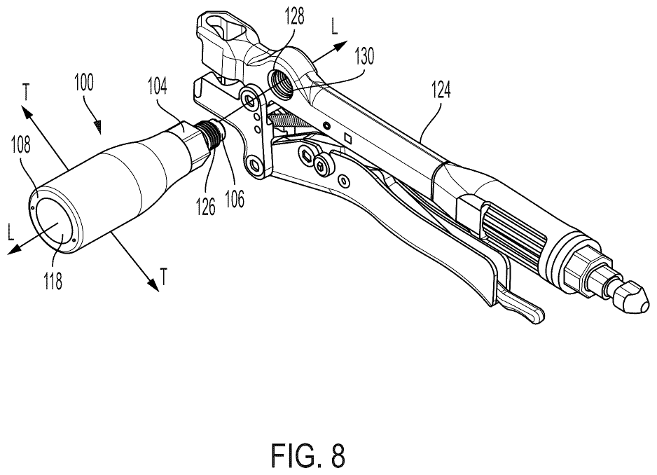

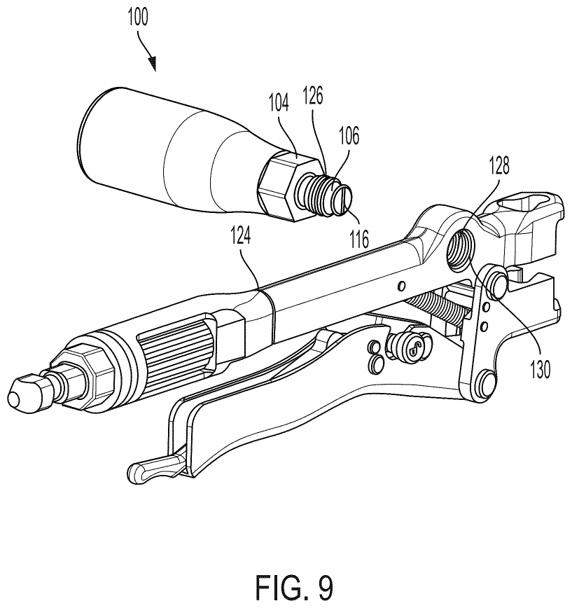

Fastener 106 may include one or more fastening mechanisms, such as threads 126 (FIG. 1), a quick connect, a taper-lock, a taper, a cylinder, a cam lock, and the like. In examples in which striking apparatus 100 is detachably coupled to extractor tool 124, the fastener may engage with a corresponding portion of extractor tool 124. For example, in examples in which fastener 106 includes threading 126 to engage with extractor tool 124, such threading 126 may engage with corresponding threading 130 within an orifice 128 of extractor tool 124 (FIGS. 8 and 9) to detachably couple striking apparatus 100 with extractor tool 124. As described throughout the specification, in examples in which threading 126 of fastener 106 engages with threading 130 of orifice 128, the striking apparatus may be attached or detached via neck 104 being turned along longitudinal axis L in a clockwise or counterclockwise direction, respectively.

Fastener 106 may include a proximally facing flat end 114 (FIG. 4), although in other examples the end portion of fastener may be other than flat shaped, such as curved shaped. In examples the flat end may include a female socket 116 shown on FIG. 4. Female socket 116 may be a linear socket in examples, although in other examples female sock may be a hex socket, a Philips socket, a Torx socket, or the like. Alternatively, the fastener may be configured as a protuberance. The protuberance may be engaged with a socket wrench, in examples. The fastener may be used to fasten (or unfasten) striking apparatus 100 from extractor tool 124. For example, the fastener (e.g., female socket, protuberance) may be used to turn the striking apparatus 100 along a longitudinal axis L of striking apparatus 100 (e.g., along longitudinal axis L of body 102). In other examples the fastener may be used to hold striking apparatus 100 in place while extractor tool 124 is turned (e.g., turned via neck 104).

The striking apparatus 100 may be manufactured via a machining process, a 3D printing process, additive manufacturing processes, a fabrication process, and the like. The fabrication process may include converting raw metal (e.g., stainless steel) into the shape of the striking apparatus 100 via a variety of fabrication processes such as cutting, bending, welding, and/or assembling processes.

Striking apparatus 100 may couple (e.g., detachably couple) to extraction tool 124. For example, striking apparatus 100 may couple to extraction tool 124 via a fastening means of striking apparats 100 coupling to an attachment mechanism of extraction tool 124. In examples, fastener 106 may include one or more fastening mechanisms, such as threads 126, a quick connect, a taper-lock, a taper, a cylinder, or a cam lock. The threading used may be a ½-20 thread dimension, although other thread dimensions may be used. In examples in which striking apparatus 100 is detachably coupled to extractor tool 124, the fastening means may engage with a corresponding portion of extractor tool 124. For example, in examples in which fastener 106 includes a threading 126 to engage with extractor tool 124, such threading 126 may engage with threading 130 within an orifice 128 of extractor tool 124 (FIGS. 8 and 9) to detachably couple striking apparatus 100 with extractor tool 124.

In examples in which threading 126 of fastener 106 engages with threading 130 of orifice 128, the striking apparatus may be further attached with implant device 124 or detached from implant device 124 via a movement of striking apparatus 100. For example, striking apparatus 100 may include a neck 104 that may be formed in many shapes. In examples neck 104 may include one or more flat surfaces, whereas in other examples neck 104 may include one or more curved surfaces. In examples neck 104 may include a combination of flat surfaces and curved surfaces. Neck 104 may include one or more protuberances. An attachment mechanism may attach to the protuberance to move the striking apparatus 100 about the longitudinal axis L. Neck 104 may include one or more orifices or inserts in which an attachment mechanism may attach to for moving the striking apparatus 100 about the longitudinal axis L. In examples, neck 104 may be square-shaped, hex-shaped (FIG. 4), circular-shaped, or the like. Neck 104 may include one or more ridges, knurlings, or protrusions, for example, to aid in the gripping of neck 104. Neck 104 may be gripped by a turning mechanism. Neck 104 may be turned along longitudinal axis L in a clockwise direction to attach (e.g., further attach) striking apparatus 100 to extraction tool 124. Neck 104 may be turned along longitudinal axis L in a counter-clockwise direction to detach (e.g., further detach) striking apparatus 100 from extraction tool 124.

Striking apparatus 100 may be coupled to extraction tool 124 prior to extraction tool 124 engaging with a medical device (e.g., an IM nail implant, knee implant, hip implant, pin, k-wire, nut, spine rod, staple, screw, and the like), although in other examples striking apparatus 100 may be coupled to extraction tool 124 after extraction tool 124 engages with a medical device (e.g., an IM nail implant, knee implant, hip implant, pin, k-wire, nut, spine rod, staple, screw, and the like). In further examples striking apparatus 100 may be coupled to extraction tool 124 at the same time extraction tool 124 engages with a medical device (e.g., an IM nail implant, knee implant, hip implant, pin, k-wire, nut, spine rod, staple, screw, and the like).

After striking apparatus 100 is attached to extraction tool 124, a force may be applied to striking apparatus 100. The force applied to striking apparatus 100 is intended to transfer to extraction tool 124. Striking apparatus 100 may be used to provide a force to extraction tool 124. The force provided to the extraction tool 124 by striking apparatus 100 may be provided by a tool, such as a hammer, mallet, and the like, exerting the force to the striking apparatus which then transfers the force to extraction tool 124. In other examples a hand may provide force to the striking apparatus 100, and the striking apparatus 100 transfers such force to the extraction tool 124.

In some examples the striking apparatus may be coupled to the medical extraction tool such that the force is received by the striking apparatus 100 along a transverse axis T of striking apparatus 100. Such force received by striking apparatus 100 may be transferred from striking apparatus 100 to extraction tool 124. For example, the force applied to the striking apparatus 100 may be longitudinal to the extraction tool 124 and the force transferred from the striking apparatus 100 to the medical extraction tool may continue in the longitudinal axis. In other examples a force may be applied to striking apparatus 100 in any number of transverse axes of striking apparatus (e.g., body 102 of striking apparatus 100), and the force transferred from the striking apparatus 100 to the extraction tool 114 may continue in any respective number of axes. In other examples a force may be applied to striking apparatus 100 along the longitudinal axis L of striking apparatus 100. The force applied to striking apparatus 100 along the longitudinal axis L of striking apparatus 100 may be transferred from the striking apparatus 100 to the extraction tool 124 along an axis perpendicular to extraction tool 124. In other examples the force may be provided to the striking apparatus in one axis, and the force from the striking apparatus may be transferred to the extraction tool 124 in another axis.

Upon the extraction tool 124 removing the medical device (e.g., IM nail implant, knee implant, hip implant, pin, k-wire, nut, spine rod, staple, screw, and the like), the striking apparatus 100 may be decoupled from extractor device 124. In examples in which fastener 106 includes threading 126 that engages with threading 130 of orifice 128, the striking apparatus may be decoupled from extraction tool 124 via neck 104 being turned along longitudinal axis L in a counterclockwise direction. In examples in which fastener 106 includes a quick connect, a taper-lock, a taper, a cylinder, or a cam lock, striking apparatus 100 may be decoupled from extraction tool 124 via detaching mechanisms known in the art.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the subject disclosure as defined by the appended claims.

I/We claim:

1. A striking apparatus for a medical extraction tool comprising:
an elongated body;
a neck extending from the elongated body; and
a fastener extending from the neck.

2. The striking apparatus of claim 1, wherein the elongated body comprises an annular distal end.

3. The striking apparatus of claim 2, wherein the elongated body further comprises a first tapering portion extending from the annular distal end and a second tapering portion extending from the first tapering portion.

4. The striking apparatus of claim 3, wherein the first tapering portion is an annular first tapering portion.

5. The striking apparatus of claim 3, wherein the first tapering portion is frustum shaped.

6. The striking apparatus of claim 3, wherein the first tapering portion is positioned about a midpoint of an overall length of the striking apparatus.

7. The striking apparatus of claim 3, wherein the second tapering portion has an overall diameter less than the first tapering portion.

8. The striking apparatus of claim 1, wherein the elongated body has an overall diameter of about 30 mm to about 33 mm.

9. The striking apparatus of claim 2, wherein the annular distal end has a wall thickness of about 9 to about 11 mm.

10. The striking apparatus of claim 1, wherein the elongated body has an overall longitudinal length of about 95 mm to about 100 mm.

11. The striking apparatus of claim 3, wherein an outer surface profile of the first tapering portion and the second tapering portion includes a point of inflection.

12. The striking apparatus of claim 1, wherein the neck includes a flat side surface.

13. The striking apparatus of claim 1, wherein the neck is square-shaped, hex-shaped, or circular-shaped.

14. The striking apparatus of claim 1, wherein the neck is configured to manipulate the striking apparatus about a longitudinal axis of the elongated body.

15. The striking apparatus of claim 1, wherein the neck includes a plurality of ridges, knurlings, and/or protrusions, and wherein the fastener comprises threads, a quick connect, a taper-lock, a taper, a cylinder, and/or a cam lock.

16. The striking apparatus of claim 1, wherein the fastener comprises a proximally facing flat end having a female socket.

17. The striking apparatus of claim 16, wherein the female socket is a linear socket, a hex socket, a Philips socket, or a Torx socket.

18. The striking apparatus of claim 1, wherein the elongated body, the neck and the fastener are a unitary part.

19. The striking apparatus of claim 1, having a longitudinal midplane and a weight of the striking apparatus is substantially balanced about the midplane.

20. The striking apparatus of claim 2, wherein the annular distal end of the elongated body comprises an aperture.

21. The striking apparatus of claim 1, wherein the elongated body is substantially hollow.

22. A medical extraction tool comprising:
an extractor body having an attachment mechanism; and
a striking apparatus comprising:
an elongated body,
a neck extending from the elongated body, and
a fastener extending from the neck,
wherein the fastener extending from the neck of the striking apparatus is configured to detachably couple with the attachment mechanism.

23. The medical extraction tool of claim 22, wherein the elongated body comprises an annular distal end.

24. The medical extraction tool of claim 22, further comprising:
a first arm extending from the extractor body and comprising a first jaw; and
a second arm pivotally connected to the first arm and comprising a second jaw.

25. The medical extraction tool of claim 23, wherein the elongated body further comprises a first tapering portion extending from the annular distal end and a second tapering portion extending from the first tapering portion.

26. The medical extraction tool of claim 25, wherein the first tapering portion is an annular first tapering portion.

27. The medical extraction tool of claim 22, wherein the attachment mechanism is an orifice.

28. The medical extraction tool of claim 22, wherein the fastener comprises threads, a quick connect, a taper-lock, a taper, a cylinder, or a cam lock.
